(19) United States
(12) Patent Application Publication
(10) Pub. No.: US 2002/0133228 A1
(43) Pub. Date: Sep. 19, 2002

(54) Adjustable intraocular lens

(76) Inventor: Edwin J. Sarver, Merritt Island, FL (US)

Correspondence Address: Michael A. Slavin, McHale & Slavin, P.A., Suite 402, 4440 PGA Boulevard, Palm Beach Gardens, FL, 33410, US

(21) Appl. No.: 10/099,204

(22) Filed: Mar. 13, 2002

Related U.S. Application Data
(60) Provisional application No. 60/275,220, filed on Mar. 13, 2001.

(51) Int. Cl. A61F 2/16

(52) U.S. Cl. 623/6.22

(57) ABSTRACT

Improved adjustable intraocular lenses are disclosed, in which the shape of the surface(s) of the lens can be modified post-operatively using manual methods or controlled pulses of laser radiation to achieve improved optical correction.

### BACKGROUND OF THE INVENTION

1. Field of the Invention:

This invention relates to improved intraocular lenses, which can be adjusted to correct spherical, cylindrical, and more general refractive errors following implantation in the phakic or aphakic eye. The intraocular lens assemblies (IOL's) of this invention may be foldable to enable implantation and removal with minimal trauma to the eye.

### DESCRIPTION OF THE PRIOR ART:

The lens of a normal human eye is situated more or less centrally behind the pupil. A normal lens is substantially symmetrical, with opposed convex surfaces. Both the lens and cornea which protects it refract incoming light to focus it on the retina. The total refraction of the eye is approximately 60 diopters. The lens supplies approximately 20 diopters of correction, while the cornea provides about 40 diopters.

When a cataract forms, the lens becomes progressively opaque and eventually has to be surgically removed, typically through a horizontal incision on the sclera or the cornea itself. Removal of the lens, however, drastically changes the focal point of the light impinging on the retina, resulting in inability to focus a clear image. Correction of focus using eyeglasses and contact lenses, or a combination thereof, is often not fully satisfactory. Eyeglasses can lead to double vision, while contact lenses require periodic replacement which may be beyond the manual skills of elderly patients in whom cataracts most frequently appear.

Prior art intraocular lenses provide a partial solution to these problems. Such lenses in the past have comprised fixed focus devices made of rigid plastic or soft, foldable materials. They are implanted via the corneal or sclera incision through which the cataract was removed, by folding the IOL (in the case of foldable designs) and inserting it into the eye behind the pupil. The IOL may then unfold and is maneuvered into place through the incision. Typically, the fixed focus IOL comprises a central optic fitted with hook-shaped haptics which attach the IOL to the walls of the posterior chamber of the eye. (Placement of the IOL within the anterior chamber is also possible in some instances). The simplest optic surface is a spherical section. IOL's also may be made having optics with asymmetrical curvature designed to correct for astigmatism. This is accomplished by creating an optic in which the curvature is different along axes 90.0 degrees apart.

Since the shapes of individual eyes vary, fixed focus IOL's must be custom made for each patient. This requires pre-operative measurement of the axial length of the eye and the curvature of the cornea; prediction of the position of the IOL in the eye after implantation, and pre-operative calculation of the proper IOL power using available formulas. Such predictions, however, are not always accurate. The shape of the eye may be changed as a result of the surgical procedure and subsequent post-operative healing process. Maeda, et al, show that the result of this post-operative shape change can affect the accuracy of traditional corneal power measurement techniques. Seitz and Langenbucher further discuss problems with accurately computing the required IOL power following corneal refractive surgery and state the prediction of the post-operative spectacle correction using traditional methods can be several diopters in error. Moreover, in the case of asymmetrically curved optic surfaces that are designed to correct for astigmatism, the desired angular orientation of the IOL within the eye may not be perfectly achieved during implantation. In fact, the desired angular orientation can change during the post-operative healing period. So can the placement of the IOL along the optical axis of the eye, which changes the effective focus of the optic. These factors are aggravated in pediatric patients, whose eyes are still changing shape as the patient grows.

Various techniques, both extracorporeal and inside the eye, have been suggested for post-operative adjustment of focus. Small adjustments of the angular orientation of the IOL may be made shortly after surgery using a needle inserted through the paracentesis incision at the corneo-scleral limbus before the IOL has been fully fixated within the capsular bag via capsular fibrosis. But, the IOL may subsequently rotate away from the revised position and at this point in time the post-operative keratometric axis typically has not yet stabilized.

Eyeglasses or contact lenses can correct for residual spherical and/or cylindrical error, thereby allowing the post-operative cataract surgery patient to achieve optimal vision. In extreme cases invasive secondary surgical procedures such as radial, astigmatic or photorefractive keratotomy may be required. Sometimes, the IOL must be replaced, or secondary IOL's must be implanted.

The types of post-operative adjustments of an IOL that may be desired are (1) changes of the axis of astigmatic correction, which is determined by the angular orientation of the IOL; (2) changes of the cylinder magnitude accompanying the astigmatism correction; (3) changes of the spherical power due to imprecise prediction of the power of the IOL for a particular patient's eye; and (4) other more general optical adjustment in which the correction in each meridian is not simply described by a sphere or cylinder. Adjustment of spherical power can be accomplished by movement of the optic along the axis of the eye; by changing the curvature of the optic, or by adjusting the index of refraction of the optic material.

Various techniques have been suggested for altering the sphero-cylindrical corrective power of IOL's. U.S. Pat. No. 5,443,506 (Garabet), for example, discloses an IOL comprising a fluid-filled lens whose focus can be changed by pumping fluids of differing refractive indices into and out of the central optic; pumping is effected by the response of various types of ionic fluids to electrical potentials generated when the ciliary body expands or contracts. U.S. Pat. No. 5,066,301 (Wiley) describes a variable focus intraocular lens comprising an envelope filled with a transparent gel in which are suspended a plurality of light-reflective particles. The orientation of the particles is said to be controllable by application of an electromagnetic or other force field, thus providing both variable spherical power and some post-operatively adjustable correction for astigmatism.

U.S. Pat. No. 4,787,903 (Grendahl) describes an IOL comprising a fresnel-type lens overlaid by a crystalline or other material that changes index of refraction when excited by electrical power or radiant energy, thus providing a post-operative adjustment function. In U.S. Pat. No. 4,816,031 (Pfoff), an IOL assembly comprising a hard PMMA (polymethyl methacrylate) optic overlain by a soft silicon optic is disclosed. The focal length of the optic assembly is adjustable by microfluid pumps that adjust the volume of clear fluid encapsulated between the PMMA optic and the silicon optic, thus changing the distance between the two optics and thereby altering the focus of the optic assembly.

U.S. Pat. No. 5,108,429 (Wiley) discloses a non-foldable IOL assembly in which a rigid hoop surrounds a fixed-focus optic. The hoop is fixed in the eye by means of external haptics; the optic is attached to the hoop by a plurality of micromotor devices illustrated as pistons which are said to be able to move the optic back and forth with respect to the hoop in response to computer-controlled electrical signals.

U.S. Pat. No. 5,203,788 (Wiley) describes a non-foldable IOL assembly in which a rigid outer ring or hoop surrounds an inner optic; the optic is operatively attached to a helical groove in the outer ring via a plurality of micromotors which are said to be operable to adjust the focus or power of the IOL assembly by changing the distance of the optic from the retina.

U.S. Pat. No. 4,994,082 (Richards, et al.) discloses an IOL assembly comprising a pair of optics hinged at one portion of their circumferences and operatively attached to the ciliary muscle at an opposing portion of their circumferences, so that expansion or contraction of the ciliary muscle changes the distance between the optics and thereby the focus of the assembly. In U.S. Pat. No. 4,790,847 (Woods), a capsule assembly surrounds an optic which is attached to the ciliary muscle. Movement of the muscle urges the optic against the anterior wall of the capsule with more or less force, thus changing the focus of the overall assembly.

U.S. Pat. No. 5,288,293 (O'Donnell) discloses an intraocular lens comprising a layer or series of layers of materials which respond to the application of laser energy, forming microfenestrations by collapsing, thus altering the front curvature of the lens. And U.S. Pat. No. 4,575,373 (Johnson) discloses a laser adjustable IOL especially designed for post-operative correction of astigmatism, in which the lens comprises an outer ring made of heat-shrinkable plastic.

The plastic is colored to permit selective absorption of laser energy. The IOL is adjusted post-operatively by focusing laser energy on parts of the outer ring, causing selective shrinkage and warping the optic to create the desired variation between curvatures along the vertical and horizontal meridians.

U.S. Pat. Nos. 5,964802 (Anello) and 5,984,962 (Anello) disclose an adjustable intraocular lens which can be adjusted following implantation using manual or remote means. The lens can alter its spherical correction by repositioning a refractive surface along the optical axis. This repositioning is accomplished by translating the lens within a 4.0 mm frame. Rotating the cylinder optic lens within its fixed frame can modify the axis of cylinder correction. The optical cylinder power is adjusted by translating the cylinder optic along the optical axis. In this scheme, post-operative adjustments are made (1) invasively and manually using an adjustment probe; (2) non-invasively using pulsed laser radiation to form shock waves which are translated into rotational mechanical force using “angled vanes”; or (3) by use of micro-motors using control and power systems as described in U.S. Pat. Nos. 5,108,429 (Wiley) and 5,066,301 (Wiley).

U.S. Pat. No. 6,013,101 (Israel) discloses an adjustable intraocular lens which uses the patient's ciliary muscle's contraction and relaxation to translate the vertical motion into a horizontal translation of the position of the lens thereby affecting a change in focusing power of the lens. This lens provides post-operative adjustment of the lens in a sense, but the extent of the adjustment is not fixed and the amount of adjustment is determined by the patient's ability to manipulate the lens via the ciliary muscle. The lens thus may be useful as a means to provide accommodation (as it was intended), but is not a viable solution to the problem addressed by the present invention.

Generally speaking, therefore, prior art approaches to achieving post-operative adjustment of the focusing capability of an IOL, therefore, have required movement of fluid into, out of or within an IOL assembly; micromotor-actuated adjustment of the position of optics operatively mounted within rigid hoops or rings; refractive index adjustment in response to externally-applied energy, or one-time, irreversible warping of an optic held inside a rigid ring by selective application of laser energy to parts of the surrounding ring.

### SUMMARY OF THE INVENTION

It is an object of this invention to provide an IOL that can be adjusted post-operatively to alter its power so as to minimize the residual optical error of the eye with respect to some refractive target not necessarily emmetropia. This correction includes spherical errors, cylinder errors, axis errors, or higher order optical aberrations.

It is a further object of this invention to provide an adjustable IOL that can be foldable, for easy implantation and explantation with minimum trauma.

It is yet another object of this invention to provide an IOL that can be adjusted post-operatively so as to alter spherical, astigmatic, or higher order optical errors that were either present prior to surgery or that may develop during the healing period following cataract or implantation surgery.

It is a related and additional object of this invention to provide methods for post-operative adjustment of the IOL's of this invention based on calculations from keratometric and other biometric measurements.

It is still another object of this invention to provide an IOL that can be adjusted post-operatively using non-invasive pulsed laser radiation, and in which such adjustment can be made repeatedly in any desired direction.

It is a further object of this invention to provide an IOL which can be adjusted post-operatively using non-invasive light transmissions to command removable fissure closures to be removed.

It is a further object of this invention to provide an adjustable IOL that can be adjusted post-operatively using surgical tools if the non-invasive adjustment mechanism fails. These and other objects of our invention are achieved by providing intraocular assemblies incorporating a fixed posterior optic surface (spherical or with a toric component) and an adjustable front surface where the surface can be adjusted by opening or closing fissures to provide regions of higher or lower optical power. These fissure openings or closings are remotely controlled using light activation. In the case of opening a fissure, a light source is modulated to command certain removable links to be removed. In the case of closing a fissure, the light source is used to shrink certain shrinkable links. In this way spherical, cylindrical, and higher order optical errors can be corrected following implantation.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1 shows an IOL based on our invention which can be adjusted post-operatively to alter the spherical, cylindrical, or higher order optical correction.

FIG. 2 shows a side view of the IOL of FIG. 2.

FIG. 3 shows the basic structure of the adjustable optical surface.

FIG. 4 shows another view of the adjustable optical surface with open and closed fissures.

FIG. 5 shows the basic mechanism for the adjustable optical surface.

FIG. 6 shows the initial and adjusted states of the adjustable surface fissures for the removable closures and shrinkable closures strategies.

FIG. 7 shows the non-invasive adjustment tool for the shrinkable closures adjustable surface.

### DETAILED DESCRIPTION OF THE PREFERRED EMBODIMENTS

Although the invention will be described in terms of a specific embodiment, it will be readily apparent to those skilled in this art that various modifications, rearrangements and substitutions can be made without departing from the spirit of the invention. The scope of the invention is defined by the claims appended hereto.

### Intraocular Lens Assembly Structure

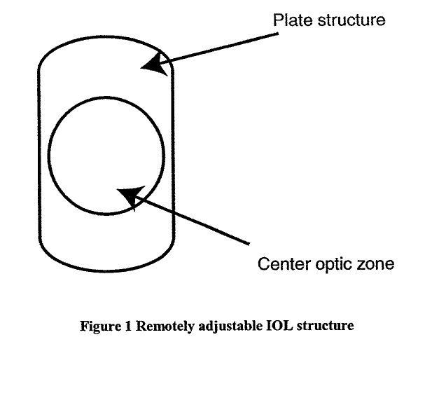

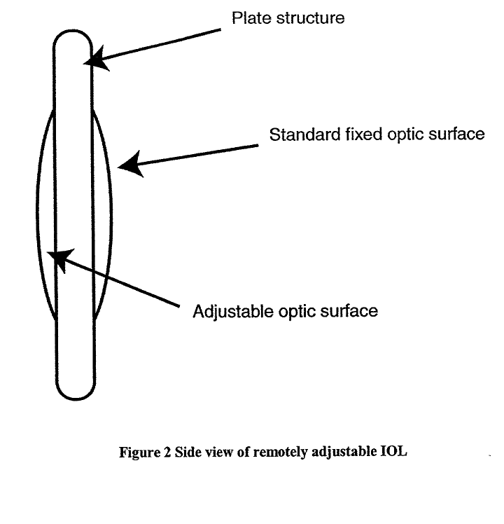

FIG. 1 illustrates an embodiment of our invention designed to allow post-operative adjustment of spherical, cylindrical, or higher order optical errors. The IOL comprises a haptic 1, preferably having the general shape of an oval or truncated oval with a major axis of approximately 10 to 11 mm and a minor axis of about 8 to 10 mm. The haptic includes a central portion which is about 5 to 7 mm in diameter and which accommodates the adjustable optic surface 2 on the front of the lens as shown in FIG. 2 and a standard fixed optic surface 3 on the back of the lens as shown in FIG. 2. The fixed optic surface can be either spherical or toric in shape. The haptic is preferably formed as a continuous piece of an optical-grade soft, biocompatible, and foldable material such as a flexible acrylic material, a hydrophilic material, or a silicone material.

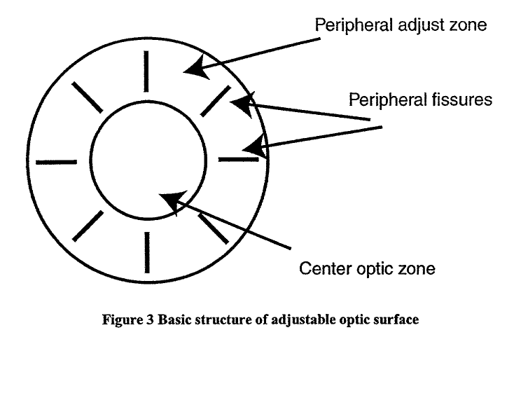

FIG. 3 illustrates the basic structure of the adjustable optical surface 2. The adjustable optical surface has a central optic zone 4 and a peripheral adjustment zone 5. Within the peripheral adjustment zone are peripheral fissures 6. These fissures are evenly spaced in the azimuth direction and are oriented in a radial direction with respect to the center of the optic surface. The number of fissures in FIG. 3 is set to eight for illustrative purposes only. In general the degree to which a surface can be adjusted increases with an increasing number of fissures. The opening or closing of these peripheral fissures is the source of the adjustability of the optical surface.

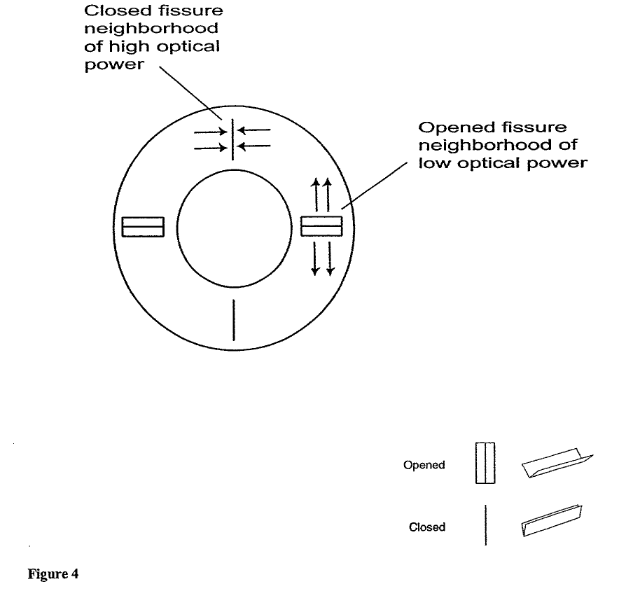

FIG. 4 illustrates opened and closed fissures in the adjustment zone of the IOL. In this figure, four fissures are shown for illustration only. The fissures oriented at 0 and 180 degrees 7 are opened and the fissures oriented at 90 and 270 degrees are closed. The opened fissures provide a lower curvature neighborhood and therefore a neighborhood with lower optical power. The closed fissures provide a higher curvature neighborhood and therefore a neighborhood with higher optical power. The particular combination of opened and closed fissures shown in FIG. 4 provides a cylinder power optical adjustment in which the vertical meridian has higher power than the horizontal meridian.

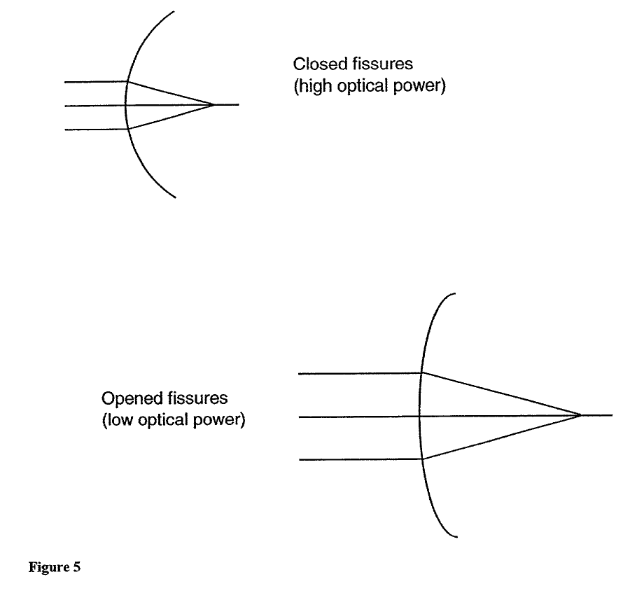

FIG. 5 illustrates the basic mechanism for the adjustable optical surface controlled by the opening of the fissures. In FIG. 5 a, we show the as implanted state of a closed fissure adjustable surface 9. This initial surface has a focal point 10. In FIG. 5 b, we show the optical result of opening the fissures to adjust the surface 11. This adjusted surface has a focal point at 12. By comparing the focal point locations 10 and 12 we illustrate that the opening of the fissures to flatten the optical surface reduces the optical power of the adjustable surface.

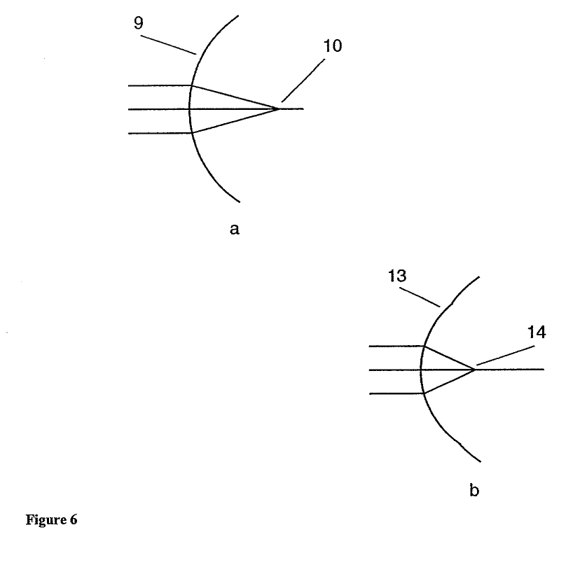

FIG. 6 illustrates the basic mechanism for the adjustable optical surface controlled by the closing of the fissures. In FIG. 6 a, we show the as implanted state of an opened fissure adjustable surface 13. This initial surface has a focal point 14. In FIG. 6 b, we show the optical result of closing the fissures to adjust the surface 15. This adjusted surface has a focal point at 16. By comparing the focal point locations 14 and 16 we illustrate that the closing of the fissures steepen the optical surface and increases the optical power of the adjustable surface.

### Optical Surface Adjustment Tools

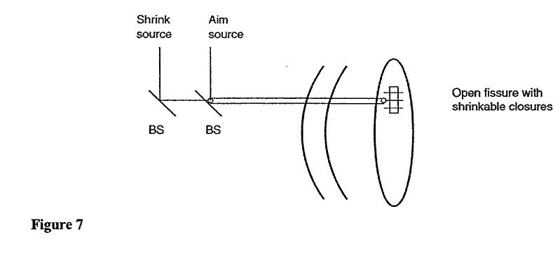

FIG. 7 illustrates the tool used for modifying the adjusting optical surface. Using the target beam 21, the user aims the adjustment tool 20 at the shrinkable or removable material of a given fissure and focuses the beam to a fine point using focusing lens 21. Then using the adjustment beam, the material is either shrunk or removed.

### Optical Calculations to Support Adjustment

The IOL is labeled as S-C/N to P, where S is the as implanted sphere in diopters, C is the as implanted cylinder in diopters, and N and P specify the amount of adjustment provided in the negative and positive direction in diopters.

### Initial Implant Selection

### For Phakic Applications

Use standard methods to compute the lens, select the best lens labeled S-C and implant. Save learning factor (LF) correction to minimize prediction error.

### For Aphakic Applications

For aphakic applications, e.g. used to correct vision following cataract surgery, use standard methods to compute the lens, select the best lens labeled S-C and implant. Save learning factor (LF) correction to minimize prediction error.

### Post-Operative Adjustment

After a suitable period post-op, determine the residual refractive error to be corrected. This residual refraction error can be measured using traditional subjective methods to obtain sphere, cylinder, and axis or using automatic methods which employ wave-front sensors as described by Liang, et. al.

### Dealing with Cataract Surgery in Later Years

If the IOL is implanted in the earlier decades, it would be expected that, due to the natural progression of the crystalline lens, cataracts will eventually develop and will require surgical intervention. The proposed procedure for this case some time in the future, will be to first explant the existing IOL. Following this explantation, perform the standard cataract removal and replacement with a new IOL computed using the algorithm above for aphakic applications.

### Other Considerations and Features

The adjustment fissures can be placed inside the lens to permit the outside of the lens to remain smooth and continuous so as to not promote cell growth. This implies a three-surface lens consisting of (1) front protection surface, (2) adjustable optic, and (3) fixed optic. Alternatively the lens could employ a front protective surface optical surface (fixed optic) and a rear adjustable optic surface.

To facilitate insertion and identification of the lens within the eye, orientation markings for the axes of a optical toric lens could be provided.

To facilitate insertion and identification of the lens within the eye, special markings or other identifiers could be made on the lens to indicate the front of the lens.

Multiple basic types of lenses could be provided in a manufacturing process. Closed fissure IOLs provide adjustment to lower power, and opened fissure IOLs provide adjustment to higher power.

To facilitate the ability to reverse an optical power adjustment, the lens could include alternating shrinkable/removable fissures.

To facilitate insertion and proper identification of the lens area during optical power adjustment, the location and types of fissures should be marked.

### Obvious Extensions of the Technology

Those of ordinary skill will understand from the foregoing disclosure that many other embodiments can be created that utilizes the features of our invention. We intend, therefore, to incorporate all such alternate embodiments and to limit our invention only as set forth in the following claims.

1. An intraocular lens assembly comprising:
a plate support structure having a substantially rounded rectangular shape of a predetermined thickness;
a fixed (non-adjustable) optical surface on one side of the plate support;
an adjustable optical surface on one side of the plate support suitable for correcting spherical, cylindrical, or other optical errors; and
a means to command the adjustable surface to be adjusted;
whereby said lens can be implanted into the eye for correction of phakic or aphakic optical errors.

2. The intraocular lens assembly of claim 1 wherein said lens is foldable to facilitate surgical implantation.

3. The intraocular lens assembly of claim 1 wherein both optical surfaces are adjustable.
